(19) United States
(12) Patent Application Publication
(10) Pub. No.: US 2014/0073915 A1
(43) Pub. Date: Mar. 13, 2014

(54) APPARATUS AND METHOD FOR VOLUMETRIC IMAGING OF BLOOD FLOW PROPERTIES

(71) Applicants: Jonghwan Lee, Boston, MA (US); David A. Boas, Winchester, MA (US)

(72) Inventors: Jonghwan Lee, Boston, MA (US); David A. Boas, Winchester, MA (US)

(73) Assignee: THE GENERAL HOSPITAL CORPORATION, Boston, MA (US)

(21) Appl. No.: 14/025,545

(22) Filed: Sep. 12, 2013

Related U.S. Application Data
(60) Provisional application No. 61/743,815, filed on Sep. 12, 2012.

(51) Int. Cl. A61B 5/026

(52) U.S. Cl. CPC A61B 5/0261
USPC 600/425; 600/479

(57) ABSTRACT

Apparatus, method and computer accessible medium can be provided for determining presence of individual scattering objects in at least one blood vessel. For example, with at least one detector arrangement, it is possible to detect interferometric radiation from at least one portion of the blood vessel(s), and provide data associated therewith. The interferometric radiation can be based on a first radiation provided from the portion at a second radiation provided from a reference. Further, with a computer arrangement, it is possible to determine the presence of the individual scattering objects in the portion of the blood vessel(s) based on the data. It is also possible to identify individual passage of the scattering objects and/or measure at least one characteristic of the passage.

### CROSS-REFERENCE TO RELATED APPLICATIONS

This application relates to and claims priority from U.S. Patent Application Ser. No. 61/743,815 filed on Sep. 12, 2012, the entire disclosure of which is incorporated herein by reference.

### STATEMENT REGARDING FEDERALLY SPONSORED RESEARCH

This invention was made with the U.S. Government support under grant numbers NIB K99-EB014879 and R01-EB000790 awarded by the National Institute of Health. Thus, the U.S. Government has certain rights therein.

### FIELD OF THE DISCLOSURE

The present disclosure relates generally to biomedical imaging, and more particularly to exemplary methods and apparatus for providing imaging (e.g., high-resolution imaging) of one or more blood flow properties in the microvasculature.

### BACKGROUND INFORMATION

Quantitative measurements of blood flow properties can play an important role in clinical disease diagnosis and animal model research. Procedures which do not require a use of contrast agents are being developed since they may be ready for in situ/clinical/pre-clinical applications. For example, Doppler Optical Coherence Tomography (“OCT”) procedure can be used for ophthalmic imaging of blood flow (see, e.g., Chen et al., 2005, “Spectral domain optical coherence tomography: Ultra-high speed, ultra-high resolution ophthalmic imaging”. Archives of Ophthalmology, 123, 1715-1720), and ultrasound, imaging is used for studies of blood volume dynamics in the whole brain (see, e.g., Mace et al., 2011, “Functional ultrasound imaging of the brain”, Nat Meth. 8, 662-664).

Unfortunately, it appears that no technique to date has facilitated label-free identification of individual red blood cell (“RBC”) flow and rapid volumetric imaging of its flow properties especially in the microvasculature such as capillaries. Doppler OCT can monitor changes in the phase of light reflected from blood flow and thereby measures the flow's axial velocity (see, e.g., Srinivasan et al., 2010b ”Quantitative cerebral blood flow with optical coherence tomography”, Opt. Express, 18, 2477-94). However, Doppler OCT and other decorrelation-based methods (see, e.g., Lee et al., 2012, “Dynamic light scattering optical coherence tomography”, Opt. Express, 20, 22262-22277) may not be suitable for measuring the other flow properties such as the RBC flux and linear density. These properties can be physiologically important, and their quantitative measurement requires identification of individual RBC passage as the flux represents how many RBCs pass for unit time with the unit of RBC/s. Doppler OCT generally does not identify individual RBC passage and only measures the axial velocity while many capillaries lie in the transverse direction. Furthermore, as RBCs generally flow one by one in capillaries, the measurement by conventional procedures of the RBC speed may not be accurate in capillaries. Further, ultrasound imaging procedures did not achieve a sufficiently high spatial resolution to identify individual RBCs (e.g., ˜8 μm in diameter).

When labeling RBCs or plasma with fluorescence, it may be possible to identify individual RBC passage in capillaries. Fluorescence two-photon microscopy can perform continuous line scanning along a capillary, and obtain stripe patterns over the capillary axis versus time space, where the slope of the stripes represents the speed of RBC flow (see, e.g., Kleinfeld et al., 1998, “Fluctuations and stimulus-induced changes in blood flow observed in individual capillaries in layers 2 through 4 of rat neocortex” Proc. Natl. Acad. Sci., 95, 15741-15746). The flux can be quantified by the number of the stripes per unit time. These measurements of the speed [mm/s]and flux [RBC/s] can lead to the linear density [RBC/mm] and hematocrit (% volume fraction). Fluorescence microscopy also can identify individual RBC flow but only within its depth of focus (see, e.g., Tomita et al., 2011, “Oscillating neuro-capillary coupling during conical spreading depression as observed by tracking of FITC-labeled RBCs in single capillaries”, Neuroimage, 56, 1001-1010). As these procedures perform either line scans along the capillary or imaging within the dun depth of focus, they may not be suitable for rapid volumetric imaging of capillary RBC flow dynamics. High-speed volumetric imaging over a large number of capillaries can be beneficial because capillaries are known to exhibit large fluctuations during baseline and diverse responses to functional activation, even with negative responses. Further, the described fluorescence-based procedures likely require exogenous contrast agents, thus limiting their in situ diagnosis applications.

In the research industry, interest in the brain's blood flow regulation has been evolving toward understanding the role of the spatio-temporal dynamics of capillary networks. In distinction to arterioles, capillaries have been reported to exhibit highly heterogeneous responses to neural activation, capillary by capillary, nearly stochastic distributions during baseline masking neural activity-induced responses within single capillaries. Therefore, a technique/procedure/system/method to measure RBC flow properties at a number of capillaries at the same time may be beneficial so that, it is possible to study the capillary flow responses in a statistical manner with high statistical significance. Furthermore, a functional study can be performed to measure the flow properties with high, temporal resolution of ˜1 s during functional activation.

According to the Mie scattering theory suggesting that 1-μm wavelength light scattering is sensitive to scatterers of 0.1-10 μm in size (see, e.g., Lee et al., 2013, “Quantitative imaging of cerebral blood flow velocity and intracellular motility using dynamic light, scattering-optical coherence tomography”, J Cereb Blood Flow Metab, 33, 819-825), large backscattering can result from RBCs. Assuming this is the case, the intrinsic scattering intensity signal of a certain position should go up and come back down when an RBC passes through the position, which in turn can facilitate a label-free identification of individual RBC passage. According to one of the objects of the present disclosure, it is possible to combine such exemplary procedure with three-dimensional (“3D”) imaging techniques that can measure the scattering intensity with sufficiently high spatial resolution can facilitate label-free volumetric imaging of blood flow properties in the microvasculature, as described in further detailed herein.

OCT procedures facilitate three-dimensional (3D) imaging of tissue structures with micrometer resolution (see, e.g., Huang et al., 1991, “Optical coherence tomography”, Science, 254, 1178-1181). It needs no contrast agents and can image at depth (up to ˜1 mm in tissue). Furthermore, such exemplary OCT procedures can simultaneously resolve ail voxels along the axial direction over the depth of focus thus improving the volumetric imaging speed by 1-2 orders of magnitude (see, e.g., Srinivasan et al., 2010a, “Rapid volumetric angiography of cortical microvasculature with optical coherence tomography”, Opt. Lett., 35, 43-5) when compared with traditional confocal and two-photon microscopes (see, e.g., Kleinfeld et al., 1998, “Fluctuations and stimulus-induced changes in blood flow observed in individual capillaries in layers 2 through 4 of rat neocortex”, Proc. Natl. Acad. Sci. 95, 15741-15746; and Kamoun et al., 2010, “Simultaneous measurement of RBC velocity, flux, hematocrit and shear rate in vascular networks”, Nat Meth. 7, 655-660).

Accordingly, it may be beneficial to address and/or overcome at least some of the current technical barriers described herein above.

### SUMMARY OF EXEMPLARY EMBODIMENTS

One of the objects of the present disclosure is to overcome certain barriers and shortcomings of the conventional arrangements and methods (including those described herein above), and provide exemplary embodiments of apparatus, systems and methods for facilitating microscopic imaging of blood flow, e.g., to measure RBC flow properties in the microvasculature with intrinsic scattering contrast.

According to an exemplary embodiment of the present disclosure, the intrinsic scattering intensity signal at a certain position fluctuates as an RBC passes through the position. Based on this determination, e.g., according to such exemplary embodiment, any technique that images the intrinsic scattering contrast with sufficiently high spatial resolution can be used for capturing individual RBC passage through capillaries, and thus quantifying the RBC flow properties. The exemplary apparatus, systems and methods according to the exemplary embodiment of the present disclosure can further utilizes such determination to provide an exemplary metric of statistical intensity variation (“SIV”) to replace the continuous monitoring of RBC passage with ensemble averaging along the capillary paths. Such further exemplary utilization can facilitate a rapid volumetric imaging of the RBC flow properties over microvasculature networks.

For example, according to another exemplary embodiment of the present disclosure, OCT exemplary procedures, systems and/or methods can be used for a continuous imaging of a cross-section through which many capillaries pass, and for capturing individual RBC passage through the capillaries and thereby for measuring the flow properties over the capillaries at the same time. As another example, exemplary rapid volumetric OCT scanning of a microvasculature network can be used for high-temporal-resolution imaging of the RBC flow properties over the capillaries consisting of the network. Such exemplary imaging procedures can be beneficial for ophthalmology diagnosis including diabetic retinopathy as the retinal capillary flow and its response to functional activation can be imaged quantitatively and in a capillary network level.

In further exemplary embodiments of the present disclosure, exemplary dynamic OCT imaging procedures can capture information regarding individual RBC passages over many vessels located at different depths at the same time. When such exemplary OCT procedure repeats continuous imaging of a cross-sectional plane through which many capillaries pass, the OCT intensity signal of a voxel located at a capillary center exhibits can peak when RBCs pass through the vessel. As each peak can represent a single RBC passage, counting the number of the peaks per unit time results in the RBC flux [RBC/s]. This exemplary measurement can be performed for each capillary passing through the imaging plane. In addition, as the peak is likely sharper when an RBC passes faster, the RBC speed [mm/s] can be determined and/or estimated from the width of the peak. By moving the cross-sectional scanning plane and repeating the above exemplary processing procedure, it is possible to obtain three-dimensional maps of the RBC flow properties over a microvasculature network. A residence time line scanning method of fluorescence two-photon microscopy has been previously described (see, e.g., Kamoun et al., 2010, ”Simultaneous measurement of RBC velocity, flux, hematocrit and shear rate in vascular networks”, Nat Meth, 7, 655-660). However, in contrast, the exemplary embodiments of the system, apparatus and method according to exemplary embodiments of the present disclosure does not require the use of contrast agents, and can monitor more than a few vessels located at different depths at the same time.

According to still another exemplary embodiment of the present disclosure, it is possible to obtain three-dimensional maps of the RBC flow properties more rapidly by using the proposed metric of SIV. In the above-described exemplary embodiment, the cross-sectional scanning plane can be anchored for a moment to capture at least several RBC passage. In contrast, based on the determination that the OCT intensity fluctuates by RBC passage, the exemplary procedure of such exemplary embodiment gathers statistical information of intensity variation along a capillary path. This collection of the statistical information can be done from, a more rapidly scanned volume data, where, e.g., only at least two scans are repeated for each cross-sectional plane. Such exemplary scanning protocol can be the one commonly used for rapid volumetric OCT angiogram (see, e.g., Srinivasan et al., 2010a, “Rapid volumetric angiography of cortical microvasculature with optical coherence tomography”, Opt. Lett., 35, 43-5). Thus, such exemplary scanning can result in obtaining the volume data of both angiogram and SIV. Mathematically, whereas the angiogram data is generally obtained from the displacement of the phase-resolved signal in the complex plane, the SIV can be obtained only from the difference in the intensity signal. There can be a number of ways to define SIV, but one definition can be:

$$SIV ( z , x , y ) ≡ \frac{{\{ I ( z , x , {t}_{2} ; y ) - I ( z , x , {t}_{1} ; y ) \}}^{2}}{\frac{1}{2} \{ {I}^{2} ( z , x , {t}_{2} ; y ) + {I}^{2} ( z , x , {t}_{1} ; y ) \}}$$

where I(z,x,t₁:y) can be the intensity data of the first B-scan over the cross-sectional plane at y, and I(z,x,t₂;y) can be the second B-scan data.

Indeed, the exemplary embodiments of the present disclosure can be implemented with, but not limited by or to, exemplary OCT systems, apparatus and/or methods.

Further, according to yet another exemplary embodiment of the present disclosure, it is possible to trace and vectorize vessel segments from either angiogram or SIV data. For a certain exemplary vectorized vessel segment, SIVs can be gathered and/or obtained along the segment path from the volume data of SIV(z,x,y). This exemplary SIV information gathered and/or obtained along the capillary segment can be used for estimating the RBC flow properties of the capillary. For example, the mean of SIV can be proportional to the RBC flax. Further statistical analysis of the SIV values (e.g., histogram) can estimate the linear density. The RBC speed can be obtained from the flux and density using the relation of (flux)=(density)×(speed). By repeating this exemplary estimation for each vectorized vessel segment, it is possible to obtain three-dimensional network maps of the RBC flow properties.

It is also possible to enhance the exemplary SIV-based estimation of the RBC flow properties by utilizing multiple time gaps. The amount that the intensity varies by RBC passage can depend on the time gap between the consecutively acquired two intensities. Using an exemplary scanning protocol according to an exemplary embodiment of the present disclosure, it is possible to obtain three or more SIV volume data with three or more different respective time gaps from the volumetric scan that repeats three B-scans for each cross-sectional plane. As such exemplary multiple-time-gap SIV data provides more plentiful statistical information, an exemplary analysis of the data can improve both the estimation accuracy and dynamic ranges.

In a further exemplary embodiment of the present disclosure, the exemplary SIV-based rapid volumetric imaging of capillary RBC flow properties can facilitate determinations of how the capillary network flow pattern varies in physiology and pathology. For example, according to one exemplary embodiment of the present disclosure, it is possible to generate and/or utilize quantitative mapping of the capillary network's RBC flow properties in the human retina, and determine how the pattern responds to various functional activation for diagnosis of various pathologies, e.g., diabetic retinopathy. In the research respective, such rapid volumetric imaging of the capillary networks flow pattern with, e.g., ˜1 s temporal resolution can facilitate monitoring of how the pattern in the cerebral cortex varies in response to somatosensory activation. Since conventional techniques did not provide simultaneous monitoring of RBC flow over hundreds of capillaries with such a high temporal resolution, such research can lead to important findings on physiological and pathological behaviors of the capillary network flow during brain's energy supply regulation, thus likely facilitating a development of various therapeutics approaches to a range of disorders of the brain.

For example, according to one exemplary embodiment of the present disclosure, apparatus, method and computer accessible medium can be provided for determining presence of individual scattering objects in at least one blood vessel.

It is possible to determine the presence of the individual red blood cells in the portion of the blood vessel e.g., using a computer arrangement, by identifying the individual scattering objects that pass through a particular position within the blood vessel or through multiple individual positions within the blood vessel. It is also possible to determine the presence of the individual scattering objects in the portion of the blood vessel without a contrast agent provided in the blood vessel. The blood vessel can be within the eye and/or the brain. The individual scattering objects can include individual red blood cells.

According to another exemplary embodiment of the present disclosure, the presence of the individual red blood cells in respective portions of multiple blood vessels can be determined based on the data. The individual scattering objects can include individual light scattering objects. The individual light scattering objects can include individual red blood cells. At least one characteristic of a plurality of the individual blood cells can be determined based on a determination of the presence thereof. Such characteristic(s) can include (i) flux, (ii) speed, (iii) hematocrit, and/or (iv) density.

In yet another exemplary embodiment of the present disclosure, it is possible to generate at least one image of the blood vessel based on the determination of the presence of the individual red blood cells with an intensity of scattering of the objects. Such image(s) of the blood vessel can include a volumetric image.

According to a further exemplary embodiment of the present disclosure, a detector arrangement which can be used to perform the detection of the interferometric radiation can obtain first and second intensities of the interferometric radiation at a first location of the multiple individual positions. It is further possible to determine differences between the first and second intensities to form first information, and generate statistical data regarding at least one characteristic of a plurality of the red blood cells based on the first information. The detector arrangement can also obtain third and fourth intensities of the interferometric radiation at a second location or a subsequent time at the first location of the multiple individual positions. It is possible, e.g., with the computer arrangement, to determine differences between the third and fourth intensities to form second information and generate the statistical data further based on the second information.

In yet another exemplary embodiment of the present disclosure, the detector arrangement can (i) obtain at least one intensity of the interferometric radiation along the blood vessel, and (ii) generate stripe pattern information representing a passage of the individual red blood cells through at least one segment of the blood vessel(s). It is possible, e.g., with the computer arrangement, to determine at least one characteristic of the plurality of the individual blood cells based on the stripe pattern information. It is also possible, e.g., with the computer arrangement, to process at least one two-dimensional image of blood vessels so as to automatically identify a position of the blood vessel(s). In one exemplary variant, it is further possible, e.g., with the computer arrangement, to (i) process at least one intensity time course associated with the blood vessel(s) so as to automatically detect peaks representing a passage of the individual red blood cells, and (ii) determine at least one characteristic of the plurality of the red blood cells based on information for the detected peaks. It is further possible, e.g., with the computer arrangement, to (i) process volumetric image data based on Hessian matrix's eigenvalues and eigenvectors of the volumetric image data to form particular information, and (ii) automatically trace and vectorize segments of a plurality of vessel based on the particular information.

In addition, according to still another exemplary embodiment of the present disclosure, the determination of the characteristic(s) of the plurality of the individual blood cells can include an estimation of at least one flow property of the individual blood cells. Such exemplary estimation can be performed, e.g., using a computer arrangement, using multiple time gaps.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF DRAWINGS

Further objects, features and advantages of the present disclosure will become apparent from the following detailed description taken in conjunction with the accompanying figures showing illustrative embodiments of the present disclosure, in which:

FIG. 1 is a schematic diagram of the spectral-domain OCT system that is used for the presented exemplary embodiments of the present disclosure;

FIG. 2A is an exemplary image of a rodent cerebral cortex through the cranial window provided using the exemplary system shown in FIG. 1;

FIG. 2B is an exemplary image of en face maximum intensity projection (“MIP”) of the 3D OCT angiogram through the depth of 0-400 μm provided using the exemplary system shown in FIG. 1;

FIG. 2C is an exemplary image of a cross-sectional slice of the OCT angiogram at the line in FIG. 2B:

FIG. 2D is an exemplary illustration of OCT intensity time courses at two selected capillary centers that are indicated by circles in FIG. 2C;

FIG. 3A is an exemplary image of the en face MIP of another exemplary 3D OCT angiogram provided using the exemplary system shown in FIG. 1;

FIG. 3B is a set of four cross-sectional slices of the OCT angiogram extracted along the dashed color lines in FIG. 3A;

FIG. 3C is an illustration of the OCT data over the capillary axis versus time, where the capillary axes are indicated by the solid color lines in FIG. 3B;

FIG. 4 is a flow diagram of an exemplary data process for estimating capillary RBC flux, speed, and density, according to an exemplary embodiment of the present disclosure;

FIG. 5A is a set of graphs of different fluctuations in the OCT intensity time courses across capillaries;

FIG. 5B is a graph of a validation that the RBC speed estimated from the exemplary Gaussian fitting process agrees with the traditional stripe pattern-based estimation performed with the same data, a part of which are shown in FIGS. 3A-3C;

FIG. 6A is an exemplary illustration of the en face MIP of still another exemplary 3D OCT angiogram with color indicating a depth from the cortical surface, as provided using the exemplary system shown in FIG. 1;

FIG. 6B is the top view of a 3D map of an exemplary capillary RBC speed based on the exemplary MIP angiogram of FIG. 6A;

FIG. 6C is the top view of a 3D map of an exemplary capillary RBC flux based on the exemplary MIP angiogram of FIG. 6A;

FIG. 6D is the top view of a 3D map of an exemplary capillary RBC density based on the exemplary MIP angiogram of FIG. 6A;

FIG. 7A is an illustration of another exemplary embodiment of the present disclosure which can be used to estimate the capillary RBC flow properties from OCT intensity time courses;

FIG. 7B is an illustration of still another exemplary embodiment of the present disclosure which can be used to estimate the capillary RBC flow properties from exemplary SIV data;

FIG. 7C is a graph illustrating a mean SIV being proportional to the RBC flux, whereas the SIVs were gathered along a certain, capillary path and then averaged, while the true flux (horizontal axis) was estimated by an exemplary embodiment of a Gaussian fitting-based procedure;

FIG. 8 is a flow diagram a further exemplary embodiment of a data process for obtaining a capillary network flux map with the exemplary SIV data according to the present disclosure;

FIG. 9A is an exemplary image of the rodent cerebral cortex through the cranial window generated using the exemplary procedure shown in FIG. 8;

FIG. 9B is a magnified image of the image shown in FIG. 9A over the area for OCT scanning;

FIG. 9C is an illustration of the exemplary en face MIP of the SIV volume data through the depth of 0-400 μm generated using the exemplary procedure shown in FIG. 8;

FIG. 10A is an illustration of the exemplary en face MIP of the exemplary tubeness volume data using the exemplary procedures according to the present disclosure;

FIG. 10B shows the SIV cross-section averaged through the segment path of FIG. 10A (top), and the exemplary mean SIV as a function of the distance from the center (bottom);

FIG. 10C is a set of illustrations of the exemplary en face and inclined views of the exemplarily vectorized capillary segments;

FIG. 11A is an illustration of IOS imaging of the somatosensory cortex of a rat for identifying the activation center generated using certain exemplary embodiments of the present disclosure; FIG. 11B is an illustration of the exemplary en face MIP of the SIV volume data over the region of interest indicated by the black box in FIG. 11A; and

FIG. 11C is a set of illustrations of a result of the exemplary functional study according to another exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

Various exemplary embodiment of the present disclosure can utilize the determination that the intrinsic scattering intensity signal fluctuates at a certain position as an RBC passes through the position. This exemplary determination can confirm that the Mie scattering-based theoretical inference that 1 ˜μm wavelength light scattering is sensitive to scatterers of, e.g., about 0.1˜10 μm in size. For example, the OCT signal that represents how much light is back-scattered from a voxel can fluctuate when an RBC passes the voxel.

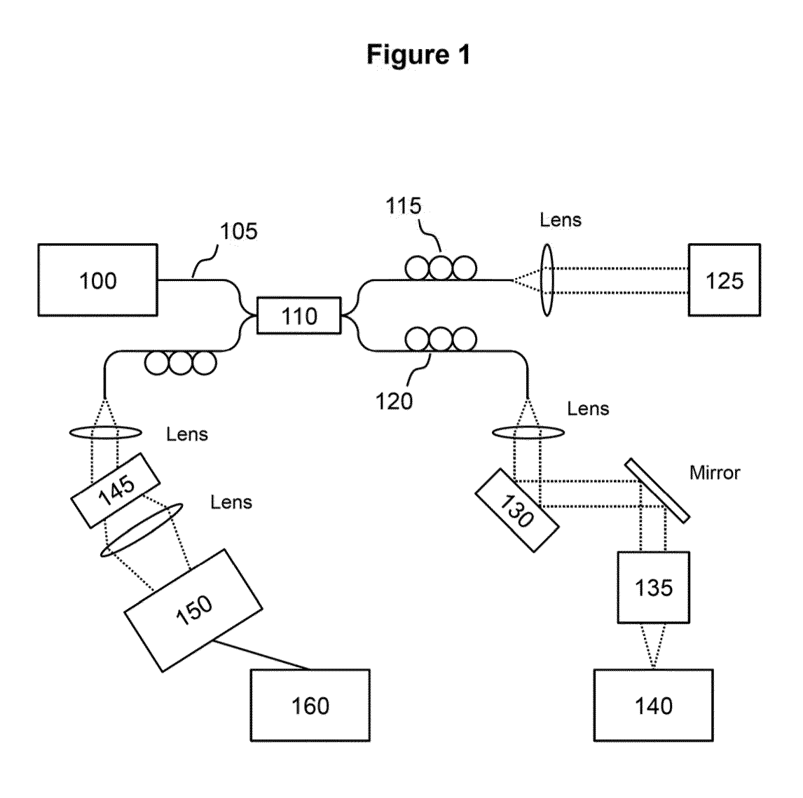

Exemplary results described herein here can be obtained using a spectral-domain OCT system, as shown in a schematic diagram of FIG. 1. Nonetheless, the exemplary embodiments of the present disclosure can be implemented by OCT systems and method, as well as various other systems and methods.

As shown in FIG. 1, broadband light (or other electro-magnetic radiation can be conveyed from the source 100 through the optical fiber 105. About half of the light beam goes to the reference path 115 while the other portion of the beam is propagated to the sample path 120. Such beam separation can be effectuated with a beam splitter/coupler 110. The beam directed to the sample path 120 can be focused on the sample 140 by the objective lens 135, and the focal spot can be moved or scanned with the galvanometer 130. The light reflected from the sample 140 can be interfered with the other light beam reflected from the reference mirror 125, at the splitter/coupler 110. The spectrum of the interfered light can be measured with a diffraction grating 145 and a line scan camera 150. The line scan camera can include a computer arrangement to perform the exemplary procedures according to exemplary embodiment of the present disclosure as described herein, and/or can be connected to a separate computer 160 perform such exemplary processing and determinations. As the spectrum fringe can be proportional to the Fourier transform of the depth profile of the sample's field reflectivity, the exemplary system, procedures and process according to certain exemplary embodiments of the present disclosure can be used to obtain, e.g., three-dimensional data of the sample's field reflectivity only by laterally scanning the sample. Exemplary system, procedures and process according to certain exemplary embodiments of the present disclosure can be performed using the spectral-domain OCT modality, as well as be embodied with any types of imaging techniques, including but not limited to swept-source OCT modality, that can be used to measure the intrinsic scattering or backscattering intensity with sufficiently high spatial resolution.

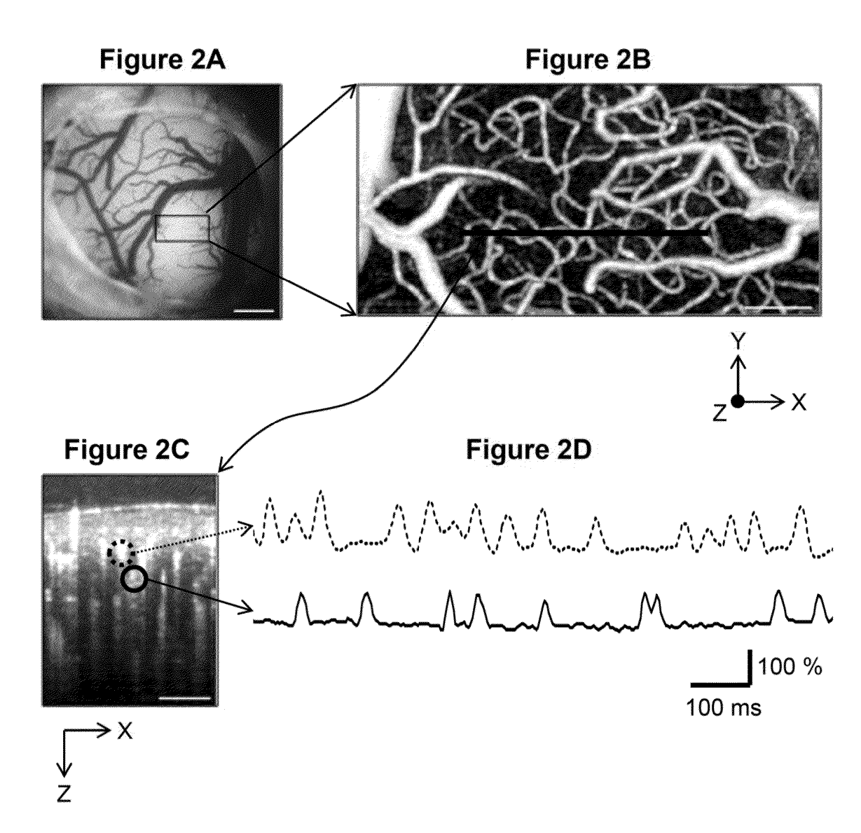

FIGS. 2A-2D shows an illustration of an exemplary experimental demonstration in which individual RBC passages are captured in the OCT intensity signals of the centers of capillaries located at different depths. In particular, FIG. 2A shows a CCD image of the rodent cerebral cortex through the cranial window. Vessels look dark as 570-nm wavelength was used for illumination and light of the wavelength is mainly absorbed by hemoglobin with RBCs. Scale bar, 500 μm. FIG. 2B shows an exemplary en face maximum intensity projection (“MIP”) of the 3D OCT angiogram through the depth of 0-400 μm. Vessels look bright as movement of RBC and plasma causes larger decorrelation of OCT signals. The angiogram was obtained over the area indicated by the solid box in FIG. 2A (scale bar, 100 μm). FIG. 2C shows a cross-sectional slice of the OCT angiogram at the line in FIG. 2B. Scale bar, 100 μm. FIG. 2D shows a graph of an exemplary OCT intensity time courses at the two selected capillary centers that are indicated by circles shown in FIG. 2C. Each peak in the time courses represents individual RBC passage. First, the peaks can be localized in space and time, with a spatial extent consistent with RBC size. While the time course at the capillary center can exhibit a number of peaks, voxels positioned in the tissue ˜10 μm far from the capillary center may not exhibit such significant peaks. Second, the intensity peaks likely do not appear from animal motion. OCT intensity has been shown to fluctuate owing to the animal's cardiac and/or respiratory motion (see, e.g., Lee et al., 2011, “Motion correction for phase-resolved dynamic optical coherence tomography imaging of rodent cerebral cortex”, Opt. Express, 19, 21258-21270). However, the RBC passage-oriented peaks appear at different moments across different capillaries (as shown in FIG. 2D), whereas motion artifact-oriented fluctuations are generally global in space. Finally, the peaks move through the capillary.

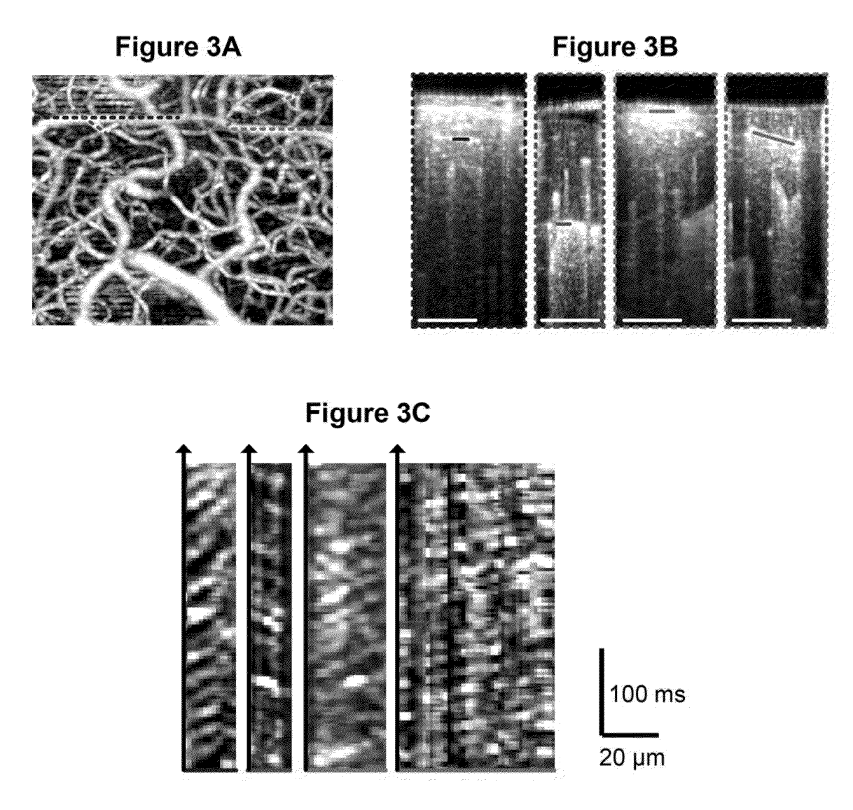

FIG. 3A illustrates an image of the exemplary en face MIP of another exemplary 3D OCT angiogram, FIG. 3B shows four cross-sectional slices of the exemplary OCT angiogram extracted along the dashed color lines as provided in FIG. 3A. The extraction lines are aligned with relatively straight capillaries. Scale bar, 100 μm. FIG. 3C shows an illustration of the exemplary OCT data over the capillary axis versus time, where the capillary axes are indicated by the solid color lines as provided in FIG. 3B. The vertical axis represents time. Stripe patterns comparable to those found in traditional two-photon line scanning methods are observed, although with no fluorescence.

As shown in FIGS. 3B and 3C, stripe patterns comparable to those found in traditional two-photon line scanning methods can be observed, although without any contrast agents. This observation of the stripe patterns strongly supports that the peaks in the signal time courses represent RBC passage through capillaries.

Various exemplary embodiments of the present disclosure as provided herein can be associated with, but not limited to, the following examples.

Cross-Sectional Imaging of Exemplary Capillary RBC Flow Properties

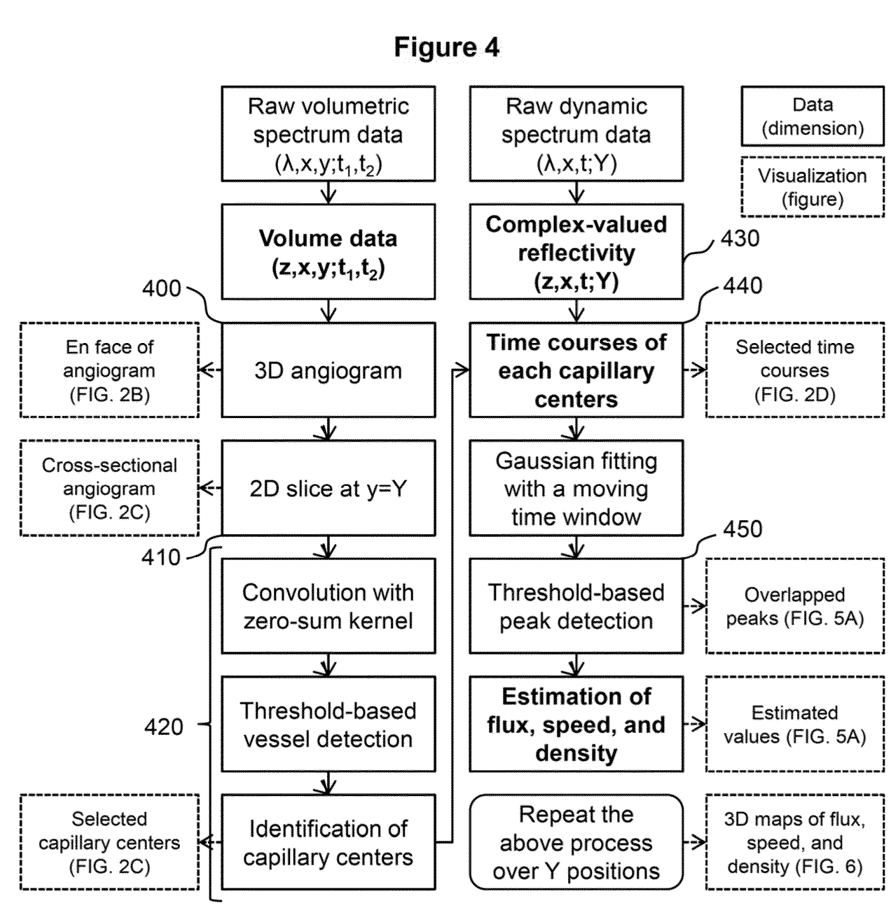

FIG. 4 illustrates a flow diagram of a process according to an exemplary embodiment of the present disclosure. The exemplary process shown in FIG. 4 includes a procedure which can be used to automatically detect the RBC passage peaks by fitting data with a moving Gaussian function. For example, the number of peaks per unit time corresponds to the RBC flux, while the mean width of the Gaussian fits can be negatively correlated with the mean RBC speed.

In particular, as shown in FIG. 4, in procedure 300, a 3D angiogram can be obtained from volumetric OCT scan data (see, e.g., Srinivasan et al., 2010a, “Rapid volumetric angiography of cortical microvasculature with optical coherence tomography”, Opt. Lett, 35, 43-5). For a cross-sectional plane at a certain Y position, a two-dimensional (“2D”) slice angiogram can be extracted (procedure 410), and an exemplary data process (procedure 420, for example) can be used for automatically identifying the centers of the capillaries passing though the cross-sectional plane. Further, B-scans can be repeated at the Y position to obtain dynamic OCT data at procedure 430. Using the position data of the identified capillary centers, the OCT intensity time courses (procedure 440) can be extracted for the capillary centers from the dynamic OCT data.

For each time course, e.g., the exemplary procedure shown in. FIG. 4 can move an 80-ms time window while fitting the data points within the window to a Gaussian function:

$$f ( {t}^{′} ) = a exp [ - \frac{{( {t}^{′} - {t}_{0} )}^{2}}{2 {b}^{2}} ] + c$$

where t₀ is the center time point of the window, and a, b, and c are fitting coefficients. Fitting can result in the values of a, b, and c and the coefficient of determination R² for each time point. Based on these exemplary values, it is possible to detect the RBC passage peaks (procedure 450) by, e.g., thresholding a>50% and R²>0.5, for example.

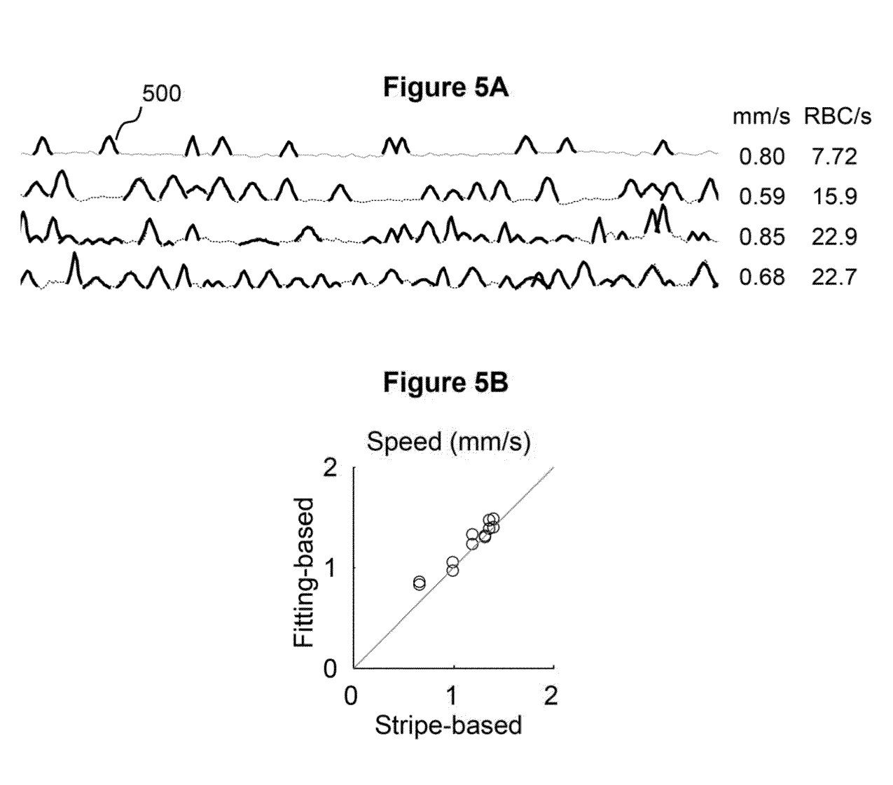

The exemplary process of FIG. 4 can adequately detect the RBC passage peaks as shown in FIG. 5A. For example, variations in the peak amplitude across RBCs may originate from various scattering profiles and orientations of RBCs. Exemplary optimum values for a and R² can vary with embodied imaging system and measurement sequence.

Such exemplary process according to the exemplary embodiment of the present disclosure (which can be performed by the exemplary system shown in FIG. 1) can estimate the RBC flux simply by counting the detected peaks per unit time. The RBC speed can be estimated, e.g., using the mean of the fitted b values, <b>:

$$v = \frac{\sqrt{{w}_{RBC}^{2} + {w}_{voxel}^{2} + {w}_{kernal}^{2}}}{2 \sqrt{2 ln 2} 〈 b 〉}$$

where w_{RBC}, wᵥₒₓₑₗ, and wₖₑᵣₙₑₗ the full-width half-maximum of the RBC, OCT voxel, and the Gaussian kernel used in the post processing, respectively.

For example, when an RBC passes a voxel with about 1 mm/s, for instance, the peak width can result from the convolution of the RBC profile with the voxel profile, leading to (w_{RBC}²+wᵥₒₓₑₗ²)^{1/2}, where all profiles can be assumed to be Gaussian. As the time course can be further convolved with a Gaussian kernel to suppress noise, the final width can become (w_{RBC}²+wᵥₒₓₑₗ²+wₖₑᵣₙₑₗ²)^{1/2}. With <b> in millisecond, with the exemplary system and method according to this exemplary embodiment, it is possible to use w_{RBC}=6.5, wᵥₒₓₑₗ=3.5, and wₖₑᵣₙₑₗ=2(2ln2)^{1/2} Δt (a Gaussian kernel with σ=Δt where Δt indicates the temporal sampling of OCT B-scans). It may be preferable but certainly not compulsory to obtain <b> by averaging over hundreds of peaks while excluding, e.g., about 10% outliers in order to suppress potential error due to RBC clumping.

The accuracy of the exemplary measurement of the capillary RBC speed was tested through comparison with traditional stripe pattern-based measurements. True values of the RBC speed were obtained from the stripe pattern (as shown in FIG. 3C) as obtained in the two-photon line scanning method (see, e.g., Kleinfeld et al, 1998, “Fluctuations and stimulus-induced changes in blood flow observed in individual capillaries in layers 2 through 4 of rat neocortex”, Proc. Natl. Acad. Sci. 95, 15741-15746). The exemplary estimation of the RBC speed based on the time courses used the exemplary data and the exemplary procedure illustrated in FIG. 4. These exemplary estimations produced results within about 9% of each other (as shown in FIG. 5B).

This exemplary embodiment assumes that RBCs have equivalently similar sizes. RBCs exhibit different orientations while flowing through capillaries, and thus different effective sizes in such imaging schemes are obtained as obtained using such exemplary embodiment. However, the effect of the different orientations on the exemplary speed estimation is negligible. This can be seen in Supplementary Figure S2 in Kamoun et al., 2010, “Simultaneous measurement of RBC velocity, flux, hematocrit and shear rate in vascular networks”, Nat Meth, 7, 655-660, which describes a residence time line scanning method. The negligible effect is again verified by the exemplary embodiment (as shown in the exemplary graph of FIG. 5B), where the exemplary estimation agrees with the exemplary stripe pattern-based estimation.

The exemplary procedure used in the exemplary method and system according to such exemplary embodiment of the present disclosure can have limited dynamic ranges of the estimation of RBC flow properties. For example, an RBC passing with about 2 mm/s can result in a peak with a width of (w_{RBC}²+wᵥₒₓₑₗ²)^{1/2}/v=3.7 ms, which can be too sharp to be accurately characterized using the temporal sampling in such exemplary procedure (Δt=4 ms). The exemplary upper limit in the dynamic range of the speed measurement using the simple definition (w_{RBC}²+wᵥₒₓₑₗ²)^{1/2}/Δt can be about 1.8 mm/s. The exemplary range of the capillary RBC speed can be about 0.1-2 mm/s. In addition, as the exemplary data process shown in FIG. 4 uses, e.g., at least five time points, the measurable flux can be limited by 1/(5Δt)=50 RBCs Nonetheless, since these dynamic ranges are functions of Δt, they can be readily extended with any type of faster imaging systems (e.g., including but not limited to currently available faster OCT systems that permit sampling times of <1 ms over large B-scans).

Exemplary Three-Dimensional Maps of Capillary RBC Flow Properties

The data acquisition and processing procedures, systems and methods according to exemplary embodiments of the present disclosure described herein can be used to obtain 3D maps of capillary RBC flow properties. For example, as one example, the exemplary procedure shown in FIG. 4 was repeated for over 96 adjacent cross-sectional planes of the sample. Capillaries in each plane were automatically identified, and their RBC speed and flux were estimated using such exemplary procedure of FIG. 4. The RBC speed, flux, and density can be estimated over a large number of positions across capillaries.

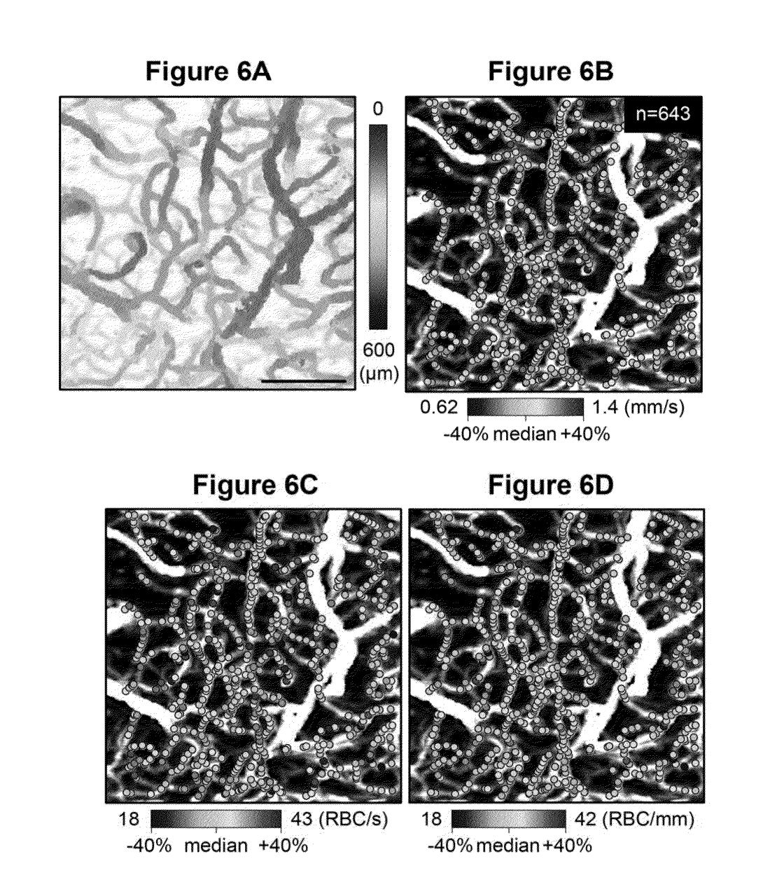

FIGS. 6A-6D show exemplary top views of such exemplary maps of capillary flow properties. The RBC linear density can be obtained with the relation of (flux)=(speed)×(density). The hematocrit also can be estimated under some reasonable assumptions about the RBC volume and plasma volume. In particular, FIG. 6B shows a top view of the 3D map of a capillary RBC speed. The estimated speed values are presented as color spots on the MIP angiogram. FIG. 6C shows atop view of the 3D map of a capillary RBC flux. FIG. 6D illustrates a top view of the 3D map of the capillary RBC density.

While conventional methods measure the capillary RBC flow properties capillary by capillary (see, e.g., Kleinfeld et al., 1998, “Fluctuations and stimulus-induced changes in blood flow observed in individual capillaries in layers 2 through 4 of rat neocortex”, Proc. Natl. Acad. Sci., 95, 15741-15746) or depth by depth (see, e.g., Tomita et al., 2011, “Oscillating neuro-capillary coupling during cortical spreading depression as observed by tracking of FITC-labeled RBCs in single capillaries”, Neuroimage, 56, 1001-1010), with the exemplary procedure, system and method according to such exemplary embodiment of the present disclosure, it is possible to measure such capillary RBC flow properties simultaneously, e.g., over many capillaries located at different depths. Such advantage facilitates a generation of exemplary 3D spatial maps such as those shown in FIGS. 6A-6D with a relatively short scan time. The exemplary embodiment was able to estimate speed, flux, and density from ˜750 locations in 384 seconds. A shorter scan time can generally lead to less contamination arising from slow variations in the human/animal physiology.

In addition, exemplary procedures, systems and methods according to exemplary embodiments of the present disclosure can measure the flow properties even when a capillary is tortuous or spans through different depths. Further, the speed estimation that can be obtained, by procedures, systems and methods according to exemplary embodiments of the present disclosure does not depend, e.g., on the direction of RBC flow as long as they use isotropic voxels. Further, procedures, systems and methods according to exemplary embodiments of the present disclosure does not require a use of any exogenous contrast agent so as to be ready for in situ or clinical applications.

Rapid Volumetric Imaging of Exemplary Capillary Network RBC Flux

With exemplary procedures, systems and methods according to further exemplary embodiments of the present disclosure, it is possible to facilitate a more rapid volumetric imaging of capillary RBC flow properties. Such exemplary procedures, systems and methods can be used to obtain and effectuate the exemplary measurement of capillary RBC flux using the exemplary metric of SIV.

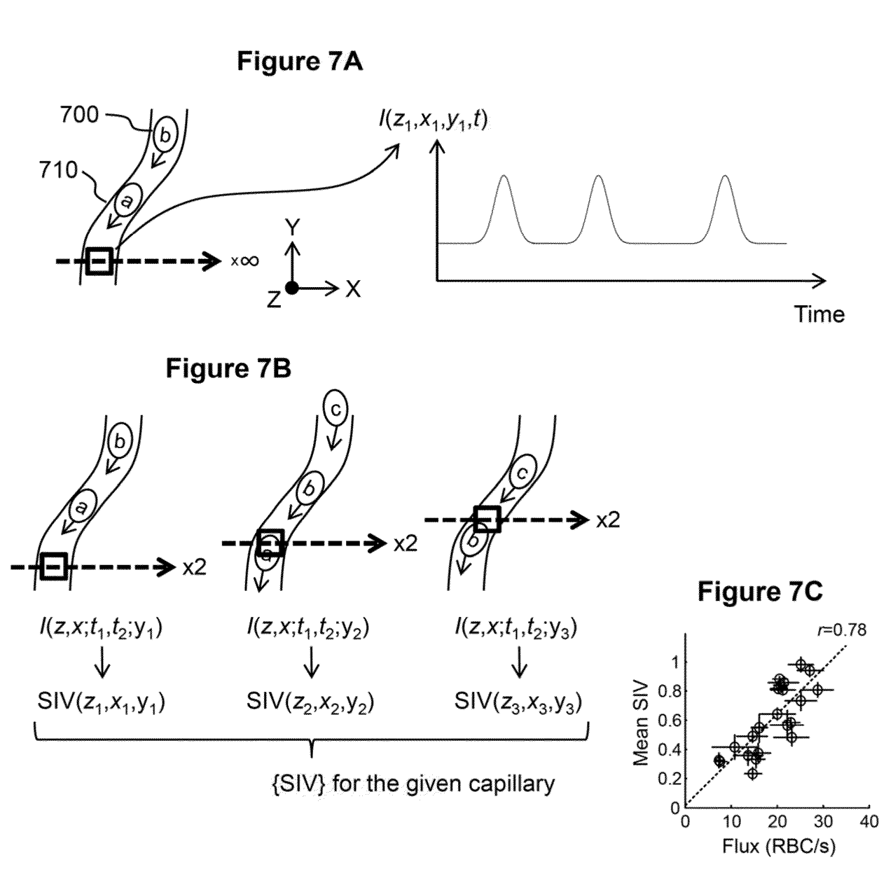

FIG. 7A shows an exemplary illustration of an exemplary implementation of a procedure according to the above-described embodiment of the present disclosure that can be used to estimates the capillary RBC flow properties from OCT intensity time courses. For example, by continuously scanning a certain cross-sectional plane (Z-X plane) that includes a certain capillary, the OCT intensity time course at the capillary center (see solid thick box) can exhibit peaks caused by the RBC passage. FIG. 7B shows an exemplary illustration of an exemplary implementation of a procedure according to another exemplary embodiment of the present disclosure that can be used to estimate the capillary RBC flow properties from SIV data. For example, by repeating only two B-scans while moving the cross-sectional scanning plane through the y-axis, it is possible to obtain statistical information of scattering intensity variation along the capillary path. The gathered exemplary information of SIV can be used for estimating the RBC flow properties of the capillary. FIG. 7C shows an exemplary demonstration in which the mean SIV can be proportional to the RBC flux. For example, the SIVs were gathered along a certain capillary path and then averaged, while the true flux (horizontal axis) was estimated by the Gaussian fitting-based procedure according to an exemplary embodiment of the present disclosure.

In particular, as depicted in FIG. 7A, the above embodiment should have the cross-sectional scanning plane anchored at a certain Y position for a moment such that individual RBCs 700 passing through a certain capillary 710 can be captured in the time course of the scattering intensity signal (as shown in the right side). Such exemplary anchoring, however, can lower the overall volumetric imaging speed, although such exemplary embodiment associated with the illustration of FIG. 7A is already faster than traditional, confocal and two-photon microscopy methods. In contrast, the exemplary procedure according to a further exemplary embodiment can repeat only two B-scans for each Y position, and rapidly can move the cross-sectional scanning plane along the y-axis, as illustrated in FIG. 7B. For example, based on the determination that the OCT signal fluctuates by RBC passage, statistical information of intensity variation can be gathered along a certain capillary path from the rapid volumetric scan data and analyzed to estimate the RBC How properties of the capillary.

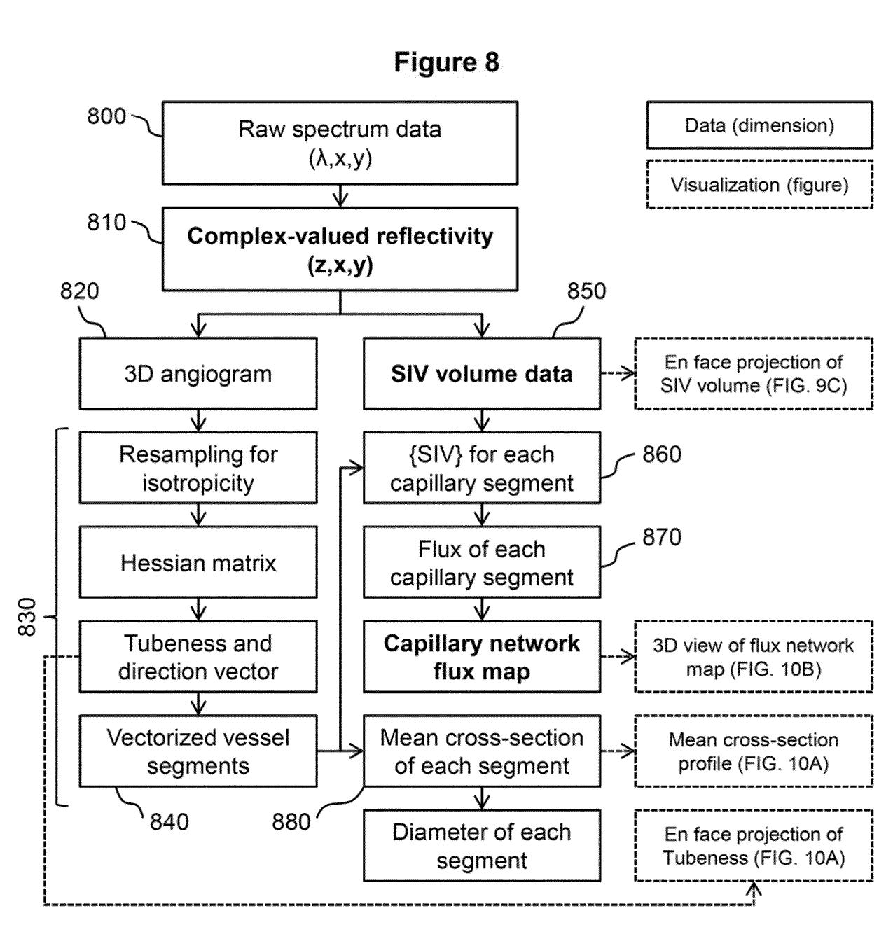

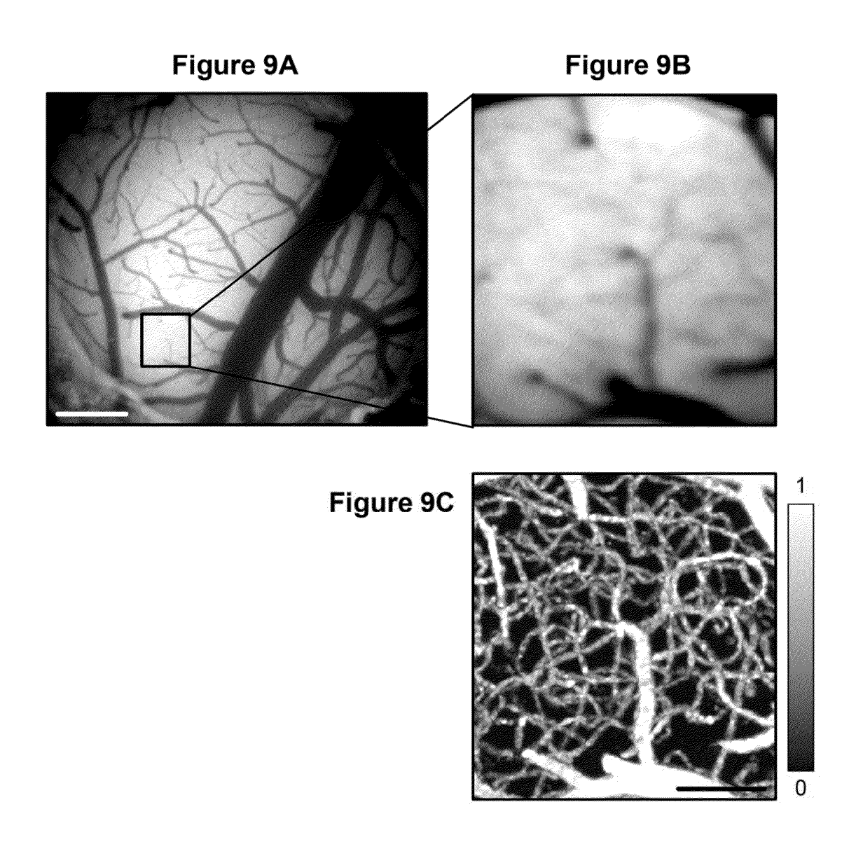

FIG. 8 shows a flow diagram of a process according to a further exemplary embodiment of the present disclosure. For example, the rapid volumetric scan with two B-scans per Y (as provided in procedure 800) can result in an OCT volume data 810. Such exemplary OCT volume data can produce both 3D angiogram (procedure 820) and SIV volume data (procedure 850). One of the possible exemplary definitions of SIV is as follows:

$$SIV ( z , x , y ) ≡ \frac{{\{ I ( z , x , {t}_{2} ; y ) - I ( z , x , {t}_{1} ; y ) \}}^{2}}{\frac{1}{2} \{ {I}^{2} ( z , x , {t}_{2} ; y ) + {I}^{2} ( z , x , {t}_{1} ; y ) \}}$$

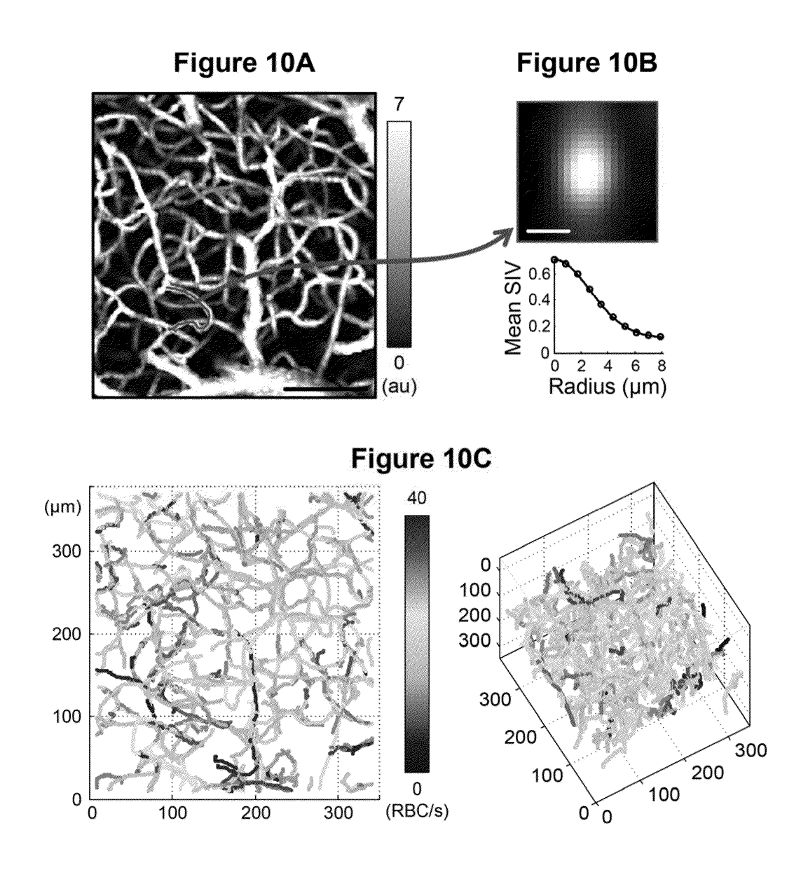

First, e.g., the 3D angiogram and/or the SIV data can be used to identity and vectorize individual capillaries, and thus to provide a mask for gathering and analyzing the SIV values along each capillary path (see procedure 830 of FIG. 8). For example, based on the image processing technique (see, e.g., Sato et al., 1998, “Three-dimensional multi-scale line filter for segmentation and visualization of curvilinear structures in medical images”, Med Image Anal. 2, 143-168), a ‘tubeness’ can be defined at every voxel using the eigenvalues of the Hessian matrix to quantify how the neighboring structure looks like a tube (see, e.g., FIG. 10A), and the eigenvectors can be used as the principal axes of the ‘tube’. As the Hessian matrix represents the second-order spatial derivatives, the tubeness can be high at the centerline of vessels, and becomes lower at the branch of vessels since the branch is less close to a tube in morphology. Such exemplary procedure 830 can be used to properly trace capillary segments and stop the tracing at their branches. In this exemplary manner, the cross-sections averaged through the segment paths can be close to 2D Gaussian patterns even when the paths were highly tortuous (see, e.g., FIG. 10B).

Then, using the exemplary information of the above vectorized vessel segments (see procedure 840) and the SIV volume data (see procedure 850), it is possible to obtain exemplary SIV values for each capillary segment (see procedure 860). Then, the obtained set of SIV values for each capillary segment, {SIV}, can be analyzed to estimate the RBC flow properties. In such exemplary manner, the exemplary process, system and procedures according to the further exemplary embodiment of the present disclosure can be used to estimate the RBC flux, FIG. 7C shows a graph which can be used to demonstrate that the mean SIV is proportional to the RBC flux. Therefore, a capillary's flux can be obtained, e.g., by averaging {SIV} of the capillary (see procedure 870), and repeating this estimation results in a capillary network flux map, as shown in FIG. 10C. Thus, the exemplary process, system and procedures according to the further exemplary embodiment of the present disclosure facilitate a measurement of the RBC flux over, e.g., hundreds of capillaries at the same time.

According to yet another exemplary embodiment of the present disclosure, it is also possible to estimate the other flow properties from {SIV}. For example, as the intensity variation is zero in principle at the moment when no RBC passes (see, e.g., an exemplary illustration of FIG. 7B), the {SIV} includes nearly zero values. Therefore, for example, the linear density can be estimated by analyzing statistical characteristics of {SIV}. When the linear density is obtained, the RBC speed can be obtained with the relation of (flux)−(density)×(speed).

Exemplary Functional Imaging of Capillary Network Flow Responses to Brain Activation

One example of possible applications of such exemplary process, system and procedures according to this exemplary embodiment of the present disclosure can be its possible use for studying how the cerebral cortex's capillary network flux pattern varies in response to functional somatosensory activation. In particular, with such exemplary process, system and procedures, it is possible to achieve a sufficiently high temporal resolution for tracing fast hemodynamic responses to functional activation. The time constant of the responses is typically ˜1 s. For example, SIV imaging was repeated so that 3D capillary network flux maps were obtained every 1.3 s during functional activation (see FIGS, 11A-11C).

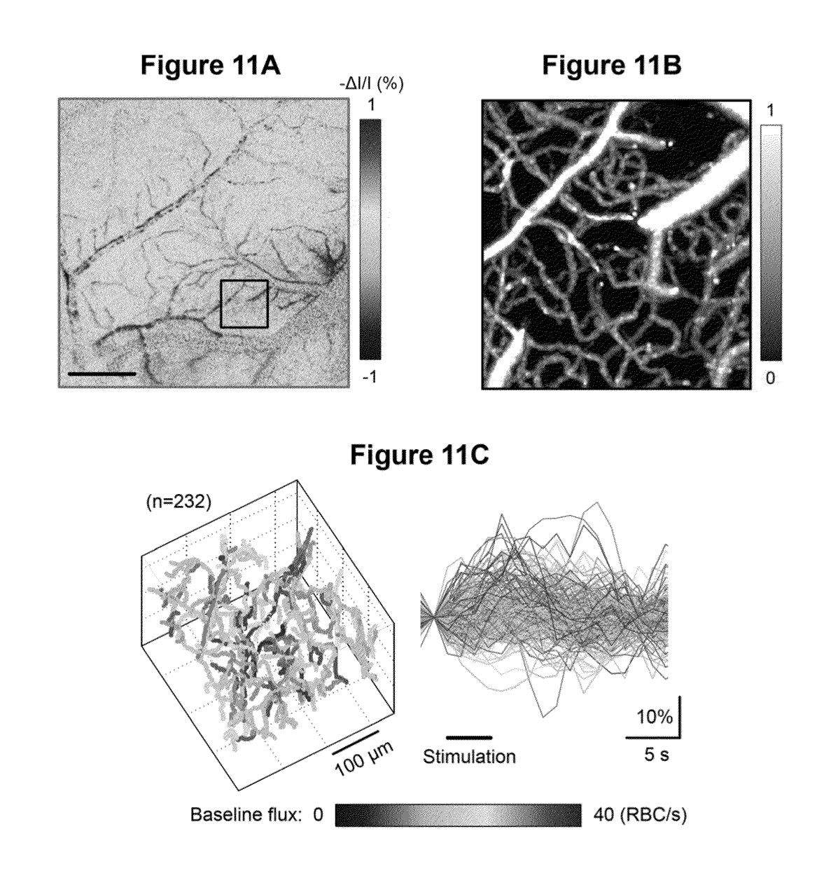

In particular, FIGS. 11A-11C show illustrations for an exemplary application of the exemplary embodiment of the present invention. The exemplary application indicates how the cerebral cortex's capillary network flow pattern varies in response to functional activation. In particular, FIG. 11A shows an illustration of an exemplary IOS imaging of the somatosensory cortex of a rat for identifying the activation center. Red color indicates increases in the blood volume in response to forepaw stimulation. Scale bar, 500 μm. FIG. 11B shows an illustration of the exemplary en face MIP of the SIV volume data over the region of interest indicated by the black box of FIG. 11A. SIV imaging was repeated every 1.3 seconds. FIG. 11B illustrates one snapshot of the time-series SIV volume data. FIG. 11C shows an exemplary result of the exemplary functional study. Based on the time-series SIV volume data, capillary segments were identified, and the RBC flux was estimated for each capillary and at each time point. This analysis enables us to trace how the flux changes over hundreds of capillaries at the same time. The capillary network flux map during the resting state is presented on the left side, and the relative RBC flux changes of the capillaries are presented on the right side, where color indicates the baseline flux of each capillary.

This exemplary experiment facilitated a tracing of relative changes in the RBC flux over hundreds of capillaries, as shown in FIG. 11C, thereby facilitating a simultaneous monitoring of RBC flow over such a large number of capillaries.

Exemplary Multiple-Time-Gap SIV Imaging

The quantitative relation between the mean SIV and the RBC flux (as shown in, e.g., FIG. 7C) can be a function of the time gap between two consecutive B-scans (i.e., t₂−t₁=δt). Therefore, it is possible, according to certain exemplary embodiments of the present disclosure, to enhance an estimation of the flow properties by employing multiple time gaps. For example, repeating three B-scans per Y position can result, in three volume data of SIV with two time gaps of δt and 2δt. The SIV values can be generally higher in the data with 2δt. The additional information of an intensity variation with different time gaps can improve the accuracy of flow property estimation when combined with a proper model of the relation between RBC flow and intensity variation.

It is further possible to implement, e.g., three or more time gaps with three or more B-scans. The above exemplary scanning protocol according to an exemplary embodiment of the present disclosure consecutively repeats three B-scans for each Y position so that the scanned Y position sequence can be 1 1 1 2 2 2 3 3 3, and so on. However, a further exemplary protocol can be provided with moving the scanning plane back and forth along the y-axis such that, for example, the scanned Y position sequence becomes 1 1 2 2 1 3 2 4 3 3 4 4 and so on. This exemplary protocol can be used to scan, e.g., three or more times for each Y position, and can result in three or more SIV volume data with three or more different time gaps of δt, 3δt, and 4δt. Other exemplary smart scanning protocols also can be provided within the scope of the exemplary embodiments of the present disclosure.

The exemplary multiple-time-gap SIV imaging procedure, system and method according to various exemplary embodiments of the present disclosure can improve the accuracy, as well as the dynamic range of the flow property measurement. The dynamic ranges of measurable RBC flux and speed with single-time-gap SIV information are functions of the time gap. Therefore, a larger dynamic range can be achieved by, e.g., combining the exemplary SIV information with more than one time gaps.

Exemplary Ophthalmic Imaging of Capillary Network Flow Dynamics

Since the exemplary systems, methods, apparatus and procedures according to exemplary embodiments of the present disclosure does not need to rely on an exogenous contrast agent, it can be also be used for in situ or clinical diagnosis. For example, such exemplary systems, methods, apparatus and procedures can be utilized and/or embodied for human ophthalmology diagnosis, and can be beneficial for diagnosis of diabetic retinopathy if the retinal capillary flow and its response to functional activation can be imaged quantitatively and in a capillary network level. Exemplary Doppler OCT procedures, systems and methods can measure the axial velocity of blood flow, but it can be difficult to quantify the RBC flux as well as the speed in capillaries, especially in those lying in the lateral direction. Further, exemplary Doppler OCT procedures, systems and methods can require at least several consecutive scans per position for gathering sequential phase information. In contrast, the exemplary systems, apparatus, method and procedures according to certain exemplary embodiments of the present disclosure utilized with the metric of SIV can require, e.g., only two B-scans so that a higher volumetric imaging speed can be obtained with therewith (e.g., the exemplary OCT method, system, modality, procedure, etc.). Such exemplary embodiments can also be used to estimate other flow properties than the speed as it is based on the determination that the OCT intensity varies with individual RBC passage. Further, the exemplary systems, apparatus, method and procedures according to various exemplary embodiments of the present disclosure can quantify RBC flow in regardless of the flow direction as long as they use isotropic voxels. Thus, the exemplary systems, apparatus, method and procedures according to the exemplary embodiments of the present disclosure can be used for ophthalmic imaging of blood flow dynamics, e.g., by only implementing software or by modifying only a small portion of hardware when needed.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated, herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination, thereof. In addition, certain terms used in the present disclosure. Including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference.

What is claimed is:

1. An apparatus for determining presence of individual scattering objects in a blood vessel, comprising:
at least one detector arrangement configured to detect interferometric radiation from at least one portion of the blood vessel and provide data associated therewith, wherein the interferometric radiation is based on a first radiation provided from the portion at a second radiation provided from a reference; and
a computer arrangement which is configured to determine the presence of the individual scattering objects in the portion of the blood vessel based on the data.

2. The apparatus according to claim 1, wherein the individual scattering objects include individual red blood cells.

3. The apparatus according to claim 1, wherein the computer arrangement determines the presence of the individual scattering objects in the portion of the blood vessel by identifying the individual scattering objects that pass through a particular position within the blood vessel.

4. The apparatus according to claim 1, wherein the computer arrangement determines the presence of the individual scattering objects in the portion of the blood vessel without a contrast agent provided in the blood vessel.

5. The apparatus according to claim 1, wherein the blood vessel is within at least one of the eye or the brain.

6. The apparatus according to claim 1, wherein the computer arrangement determines the presence of the individual scattering objects in the portion of the blood vessel by identifying the individual scattering objects that pass through multiple individual positions within the blood vessel.

7. The apparatus according to claim 2, wherein the computer arrangement further determines the presence of the individual red blood cells in respective portions of multiple blood vessels based on the data.

8. The apparatus according to claim 1, wherein the individual scattering objects include individual light scattering objects.

9. The apparatus according to claim 8, wherein the individual light scattering objects include individual red blood cells.

10. The apparatus according to claim 9, wherein the computer arrangement is further configured to determine at least one characteristic of a plurality of the individual blood cells based on a determination of the presence thereof.

11. The apparatus according to claim 10, wherein the at least one characteristic includes at least one of (i) flux, (ii) speed, (iii) hematocrit, or (iv) density.

12. The apparatus according to claim 1, wherein the computer arrangement is further configured to generate at least one image of the blood vessel based on the determination of the presence of the individual red blood cells with an intensity of scattering of the objects.

13. The apparatus according to claim 12, wherein the at least one image of the blood vessel is a volumetric image.

14. The apparatus according to claim 2, wherein the detector arrangement is further configured to obtain first and second intensities of the interferometric radiation at a first location of the multiple individual positions, and wherein the computer arrangement is further configured to determine differences between the first and second intensities to form first information and generate statistical data regarding at least one characteristic of a plurality of the red blood cells based on the first information.

15. The apparatus according to claim 14, wherein the detector arrangement is further configured to obtain third and fourth intensities of the interferometric radiation at a second location or a subsequent time at the first location of the multiple individual positions, and wherein the computer arrangement is further configured to determine differences between the third and fourth intensities to form second information and generate the statistical data further based on the second information.

16. The apparatus according to claim 10, wherein the detector arrangement is further configured to (i) obtain at least one intensity of the interferometric radiation along at least one vessel, and (ii) generate stripe pattern information representing a passage of the individual red blood cells through at least one segment of the blood vessel, and wherein the computer arrangement is further configured to determine at least one characteristic of the plurality of the individual blood cells based on the stripe pattern information.

17. The apparatus according to claim 10, wherein the computer arrangement is further configured to process at least one two-dimensional image of the blood vessel so as to automatically identify a position of the blood vessel.

18. The apparatus according to claim 10, wherein the computer arrangement is further configured to (i) process at least one intensity time course associated with the at least one vessel so as to automatically detect peaks representing a passage of the individual red blood cells, and (ii) determine at least one characteristic of the plurality of the red blood cells based on information for the detected peaks.

19. The apparatus according to claim 14, wherein the computer arrangement is further configured to (i) process volumetric image data based on Hessian matrix's eigenvalues and eigenvectors of the volumetric image data to form particular information, and (ii) automatically trace and vectorize segments of a plurality of blood vessels based on the particular information.

20. The apparatus according to claim 10, wherein the determination of the at least one characteristic of the plurality of the individual blood cells includes an estimation of at least one flow property of the individual blood cells, and wherein the computer arrangement is configured to estimate the of at least one flow property using multiple time gaps.

21. A process for determining presence of individual scattering objects in at least one blood vessel, comprising;
detecting interferometric radiation from at least one portion of the blood vessel and provide data associated therewith, wherein the interferometric radiation is based on a first radiation provided from the portion at a second radiation provided from a reference; and
with a computer arrangement, determining the presence of the individual scattering objects in the portion of the blood vessel based on the data.

22. A non-transitory computer medium which includes instructions thereon for determining presence of individual scattering objects in at least one blood, vessel, wherein, when the instructions are executed by a computer arrangement, the computer arrangement is configured to perform procedures comprising:
causing a detection of interferometric radiation from at least one portion of the blood vessel and provide data associated therewith, wherein the interferometric radiation is based on a first radiation provided from the portion at a second radiation provided from a reference; and
determining the presence of the individual scattering objects in the portion of the blood vessel based on the data.
